(19) United States
(12) Patent Application Publication
(10) Pub. No.: US 2013/0095151 A1
(43) Pub. Date: Apr. 18, 2013

(54) BROAD SPECTRUM SUNSCREEN COMPOSITION COMPRISING 2 HYDROXY SULFOBETAINE OF CINNAMIDOALKYL AMINE

(71) Applicants: Arun Harachandra Jawale, Maharashtra (IN); Vaishali Amol Jumde, Maharashtra (IN); Archana Kishor Desai, Shivsrushti (IN)

(72) Inventors: Arun Harachandra Jawale, Maharashtra (IN); Vaishali Amol Jumde, Maharashtra (IN); Archana Kishor Desai, Shivsrushti (IN)

(73) Assignee: GALAXY SURFACTANTS LTD., Maharashtra, Navi Mumbai (IN)

(21) Appl. No.: 13/704,481

(22) PCT Filed: Jun. 17, 2011
(86) PCT No.: PCT/IB11/01371

§ 371 Date: Dec. 14, 2012

(30) Foreign Application Priority Data
Jun. 17, 2010 (IN) 2917/MUM/2009

(51) Int. Cl. A61K 8/46

(52) U.S. Cl. CPC A61K 8/466
USPC 424/400; 424/59; 424/60

(57) ABSTRACT

A photostable cosmetic composition for protection against UVA and UVB radiations of wavelengths between 280 and 400 nm, which comprises, in a cosmetically acceptable vehicle:
(i) at least one UVA-sunscreen agents absorbing predominantly in the UVA-range;
(ii) at least one UVB-sunscreen agents absorbing predominantly in the UVB-range;
(iii) at least 0.1% by weight of a 2-hydroxy sulfobetaine of cinnamidoalkyl amine compound.

### TECHNICAL FIELD OF THE INVENTION

The present invention relates to sunscreen compositions having high UV-A protection. In particular, it relates to sunscreen compositions, comprising 2-hydroxy sulfobetaine of cinnamidoalkyl amine compound, having higher UV-A protection and enhanced photo stability.

### BACKGROUND AND PRIOR ART

It is well known that ultraviolet radiation (light) having a wavelength from about 280 nm to about 320 nm (UV-B) is harmful to human skin, causing sunburns that are detrimental to the development of a good sun tan and accounts for severe damages like skin cancer. For these reasons, as well as for aesthetic reasons, there is an increasing demand for a means of controlling this natural tanning in order to thereby control the colour of the skin. This UV-B radiation must be screened from the skin. It is also important to have a protection from the rays of the region between about 320 nm and about 400 nm, the UV-A region, since the rays of this region can also cause damage like photoageing and wrinkles. For a long time it was incorrectly assumed that the long-wave UV-A radiation with a wavelength between 320 nm and 400 nm has only a negligible biological action and that, accordingly, the UV-B rays are responsible for most light-induced damage to human skin. However, in the meantime numerous studies have shown that UV-A radiation is much more harmful than UV-B radiation with regard to the triggering of photodynamic, specifically phototoxic, reactions and chronic changes in the skin. Moreover the UV-B noxious action can be enhanced by the presence of UV-A (Willis et al.: Journal of Investigative Dermatology vol. 59, 416, 1972).

Thus, it has inter alia been found that even UV-A radiation, under quite normal everyday conditions, is sufficient to damage, within a short time, the collagen and elastin fibres, which are of essential importance for the structure and strength of the skin. This results in chronic light-induced changes in the skin—the skin “ages” prematurely. The clinical manifestation of light-aged skin includes, for example, wrinkles and lines, and also an irregular, furrowed relief. In addition, the areas affected by light-induced skin ageing can have irregular pigmentation. The formation of brown spots, keratoses and even carcinomas or malignant melanomas is also possible. Skin which has been prematurely aged as a result of everyday UV stress is further characterized by a lower activity of the Langerhans' cells and slight, chronic inflammation.

As a result of the abovementioned hazards associated with sunlight exposure, the general public's interest in the sun protection product market has grown considerably. A wide variety of sunscreen actives have been used in personal care products. It is desirable that the sunscreen active or active system provide broad spectrum UV protection, particularly protection against both UV-A and UV-B radiation.

Consumers consider many factors when purchasing a sunscreen product, such as, the sunburn protection factor (SPF), how durable the product is after applying it over the skin, the shelf life of the product, product form (i.e., lotions, gels, creams, and sprays) and product cost. However, they fail to consider one more important property of sunscreen product is its effective and efficient protection against UVA radiation.

High SPF, reflecting the protection against UVB rays, as well as high protection against UVA rays is the foremost criteria now watched and demanded by the consumers. To establish a high protection, it is necessary to incorporate UV filters into all phases of a formulation and for this the solubility of the filter substances in the oil and water phases is of decisive importance. Most UV-filters are oil-soluble e.g. Butyl Methoxydibenzoylmethane, Ethylhexyl Methoxycinnamate, Octocrylene, and 4-Methylbenzylidene Camphor. There are only few water-soluble UV filters like, Benzophenone-4 and salts of Phenylbenzimidazole Sulfonic Acid.

Ethylhexyl Methoxycinnamate and the salts of Phenylbenzimidazole Sulfonic Acid, are among the oil-soluble and water-soluble UV-filters respectively which are effective and widely used UV-B filters and their use as UV-filters has already been reported in patents. Salts of Phenylbenzimidazole Sulfonic Acid is described in German Reichspatent No. 676 103.

U.S. Pat. No. 5,961,961 assigned to Chesebrough-Pond's USA Co., reports enhancement of the photoprotective effect by utilizing relatively large particle size Titanium Dioxide coupled with an organic sunscreen agent. Representative organic sunscreen agents include Benzophenone-3, Octyl Salicylate, Ethylhexyl Methoxycinnamate and 2-Phenylbenzimidazole-5-Sulphonic Acid.

As described in the beginning, the crucial aspect that is now considered by the consumers is the UV-A protection of the sunscreen formulations. From effectiveness and cost point of view, these formulations should have high UV-A protection along with good SPF. However, in all cases of the prior art where Phenylbenzimidazole Sulfonic Acid and Ethylhexyl Methoxycinnamate is used, the effect achieved falls a long way short of that which is desired.

Yet another vital aspect which is considered by the cosmetic and sunscreen manufacturers is photostability of the formulations.

Dibenzoylmethane compounds are one class of sunscreen compounds which provide broad spectrum UV protection and are approved for global use. Butyl Methoxydibenzoylmethane (Avobenzone) is the widely used dibenzoylmethane compound known to accomplish the requirement of UV-A protection. It is the most frequently used UV-A filter world-wide. Unfortunately, it tends to photodegrade upon exposure to UV radiation thereby reducing their UV-A efficacy. However, since it is a very desirable component of many sunscreening products, considerable effort has been devoted to studies of these instabilities. Combinations of some other sunscreening active agents with Avobenzone have been reported to enhance the photostability of Avobenzone, while combinations with certain other ingredients such as Ethylhexyl Methoxycinnamate reportedly decreases the avobenzone photostability due to a bimolecular reaction between the two photoactive compounds leading to the rapid photodegradation of both.

U.S. Pat. Nos. 5,576,354 and 5,587,150 both assigned to L′Oreal, describe the photostabilization of Avobenzone in sunscreen compositions by adding a Diphenylacrylate sunscreen ingredient, such as Octocrylene, in molar ratios of Diphenylacrylate to Avobenzone at least 0.8.

U.S. Pat. No. 6,033,649 assigned to Roche Vitamins, describes the same composition as claimed by above given L′Oreal's patents except the molar ratios of Diphenylacrylate to Avobenzone is less than 0.8.

U.S. Pat. No. 5,827,508 assigned to P&G, reports that Avobenzone can be made to have improved chemical stability and photostability in a sunscreen product formulation by incorporation of surface-treated Zinc Oxide particles, silicone-treated Zinc Oxide being particularly useful for this purpose.

U.S. Pat. No. 5,985,251 assigned to Roche Vitamins, teaches that stable sunscreen compositions can be prepared to contain Avobenzone, a Diphenylacrylate or Benzylidene Camphor derivative and a water-soluble p-Methoxycinnamate.

U.S. Pat. No. 6,071,501 assigned to P&G, states that combinations of Dibenzoylmethane derivatives and Ethylhexyl p-Methoxycinnamate are not photostable, unless the molar ratio of the Methoxycinnamate to the Dibenzoylmethane is in the range of 0.15:1 to 1:1.

U.S. Pat. No. 6,090,369 assigned to Stewart, proposes a stable sunscreen composition that contains Avobenzone, Ethylhexyl Methoxycinnamate and either Titanium Dioxide or Zinc Oxide; Octocrylene is an optional component.

U.S. Pat. No. 5,989,528 assigned to P&G, have disclosed a sunscreen composition with high stability comprising a safe and effective amount of Dibenzoylmethane active (e.g. Avobenzone), a safe and effective amount of stabilizing agent (e.g. Octocrylene), a safe and effective amount of a UV-B sunscreen (e.g. 2-Phenylbenzimidazole-5-Sulfonic Acid, Zinc Oxide, Titanium Dioxide, etc.) and a suitable carrier.

U.S. Pat. No. 7,309,481 assigned to Tanning Research, discloses a photostable sunscreen composition comprising a triplet combination of Avobenzone, Octocrylene and Oxybenzone and containing no diesters or polyesters of naphthalene dicarboxylic acid and substantially free of any optimizing agents such as diols and polyols.

U.S. Pat. No. 7,244,416 and US Patent Application Publication No 2007/189993 both assigned to Schering-Plough, relates to Zinc Oxide and Avobenzone-containing sunscreen compositions which are stabilized against Avobenzone photodegradation by the inclusion of Phenylbenzimidazole Sulfonic Acid.

US Patent Application Publication No. 2011/0052516 assigned to DSM, discloses that a photostable and high SPF composition can be achieved by combining at least 4.5 wt.-% of Butyl Methoxydibenzoylmethane with at least 2 wt.-% of polysilicones-15 and at least 2 wt. % Phenylbenzimidazole Sulfonic Acid in the presence of a phosphate ester surfactant.

US Patent Application Publication No. 2009/0155194 assigned to Schering-Plough, discloses a photostable sunscreen composition comprising Avobenzone, Zinc Oxide and phosphate-based emulsifiers (e.g. potassium cetyl phosphate).

Prolonged protection against the UV-A radiation is very important as evidenced by the numerous approaches that have been taken to improve Avobenzone photostability. Thus, it is always desired to improve the performance and stability characteristics of sunscreening products comprising especially Avobenzone. However, both Phenylbenzimidazole Sulfonic Acid and Ethylhexyl Methoxycinnamate compounds have not been able to cater to this desire so far.

Further the Phenylbenzimidazole Sulfonic Acid itself is very insoluble in water (0.25% w/w); therefore it must be neutralized with a hard base, such as for example but not limited to sodium hydroxide or potassium hydroxide, or with amines, such as for example but not limited to triethanolamine or aminomethylpropanol or trishydroxyaminomethane, in order to render the UV filter soluble in a cosmetic or pharmacological preparation. Importantly, if Phenylbenzimidazole Sulfonic Acid is not sufficiently neutralised it will form crystals in the cosmetic or pharmacological preparation rendering it less effective and giving the preparations an unacceptable sandy or grainy feel. Crystal formation starts at pH 7.0; generally, the pH of a cosmetic formulation comprising Phenylbenzimidazole Sulfonic Acid has to remain above 6.8 in order to prevent the free acid from recrystallising (W. Johncock, Cosmetic & Toiletries Magazine, September 1999, pages 75-82). This restriction has prevented the use of Phenylbenzimidazole Sulfonic Acid in formulations with pH at or below 7.0, in particular in the many cosmetic or pharmacological preparations formulations with a pH from 5.5 to 7.0 and 6.0 to 7.0.

Thus, the overall challenges that still exist which need to be addressed are:
1. to provide a cosmetic or sunscreen or dermatological composition having higher UVA protection; and
2. to enhance the stability of UV-sensitive ingredients like Avobenzone and to provide photostable cosmetic and dermatological compositions whose activities and properties are retained over a long time.

The inventors of the present invention have surprisingly found that the introduction of 2-hydroxy sulfobetaine of cinnamidoalkyl amine compound into sunscreen formulations addresses the above challenges.

It has been found that both Ethylhexyl Methoxycinnamate and 2-hydroxy sulfobetaine of cinnamidoalkyl amine compounds have similar UV absorption; the later enhances the UVA protection better than the former. Moreover, on the contrary, Ethylhexyl Methoxycinnamate destabilizes the system and decrease the UVA protection.

Unexpectedly it has been found, that sunscreen compositions containing 2-hydroxy sulfobetaine of cinnamidoalkyl amine compound provides enhanced UVA protection.

Also, the introduction of 2-hydroxy sulfobetaine of cinnamidoalkyl amine compound enhances the photostability of Avobenzone in presence of UVB agents.

### OBJECT OF THE INVENTION

It is an object of the present invention to provide an efficient photostable UV protective composition.

It is another object of the present invention to provide a broad spectrum UV protective composition which shields from both UVA and UVB radiation

It is another object of the present invention to provide such a composition that can provide improved high UVA protection.

It is another object of the present invention to provide an enhanced photostable composition comprising UVA and UVB compounds.

It is another object of the invention to provide a photostable composition providing enhanced stability to UVA compounds.

It is another object of the present invention to provide the photostable composition in the form of creams, lotions, milks, mousses, gels, tonics, sticks, and sprays.

### SUMMARY OF THE INVENTION

According to one aspect, the present invention provides a photostable cosmetic composition for protection against UVA and UVB radiation of wavelengths between 280 and 400 nm, which comprises, in a cosmetically acceptable vehicle
(i) at least one UVA-sunscreen agents selected from the group consisting of Butyl Methoxydibenzoylmethane/Avobenzone, Benzophenone-3, Benzophenone-4, Benzophenone-5, Disodium Phenyl Dibenzimidazole Tetrasulfonate, Drometrizoletrisiloxane, Terephthalyidene Dicamphor Sulfonic Acid, bis-Ethylhexyloxyphenol Methoxyphenyl Triazine, Methylene bis Benzotriazolyl Tetramethylbutylphenol, and Diethylaminohydroxybenzoylhexylbenzoate in the range 0.5 to 5%;
(ii) at least one UVB-sunscreen agent selected from the group consisting of Ethylhexyl Salicylate, Homosalate, Isoamyl p-Methoxy cinnamate, Octocrylene, 4-Methylbenzylidene Camphor, 3-Benzylidene camphor, Benzylidene Camphor Sulfonic Acid, Camphor Benzalkonium Methosulfate, Polyacrylamidomethyl Benzylidene Camphor, Ethylhexyl Triazone, Diethylhexyl Butamido Triazone and Polysilicon-15 in the range 0.5 to 10%; and
(iii) at least 0.1% by weight of a 2-hydroxy sulfobetaine of cinnamidoalkyl amine compound having general formula:

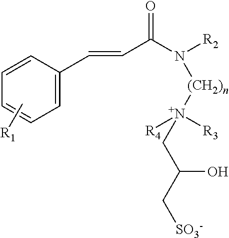

wherein,
R₁ is a substituent, selected from hydrogen, halo, —OH, —NH₂, —NO₂, —OCH₃, —N(CH₃)₂, alkyl groups containing from 1 to 6 carbon atoms, alkoxy groups containing from 1 to 6 carbon atoms, alkylamino or N,N-dialkylamino groups containing from 1 to 6 carbon atoms;
R₂ is selected from hydrogen, alkyl group containing from 1 to 12 carbon atoms;
R₃ and R₄ are independently selected from benzyl, alkyl group containing from 1 to 12 carbon atoms,
n is an integer from 1 to 6,
with the proviso that the UVB sunscreen agents exclude Ethylhexyl Methoxycinnamate and Phenylbenzimidazole Sulfonic Acid.

### DETAILED DESCRIPTION OF THE INVENTION

The present invention provides a photostable cosmetic composition for protection against UVA and UVB radiation of wavelengths between 280 and 400 nm, which comprises, in a cosmetically acceptable vehicle
(i) at least one UVA-sunscreen agents selected from the group consisting of Butyl Methoxydibenzoylmethane/Avobenzone, Benzophenone-3, Benzophenone-4, Benzophenone-5, Disodium Phenyl Dibenzimidazole Tetrasulfonate, Drometrizoletrisiloxane, Terephthalyidene Dicamphor Sulfonic Acid, bis-Ethylhexyloxyphenol Methoxyphenyl Triazine, Methylene bis Benzotriazolyl
Tetramethylbutylphenol, and Diethylaminohydroxybenzoylhexylbenzoate in the range 0.5 to 5%;
(ii) at least one UVB-sunscreen agent selected from the group consisting of Ethylhexyl Salicylate, Homosalate, Isoamyl p-Methoxy cinnamate, Octocrylene, 4-Methylbenzylidene Camphor, 3-Benzylidene camphor, Benzylidene Camphor Sulfonic Acid, Camphor Benzalkonium Methosulfate, Polyacrylamidomethyl Benzylidene Camphor, Ethylhexyl Triazone, Diethylhexyl Butamido Triazone and Polysilicon-15 in the range 0.5 to 10% and
(iii) at least 0.1% by weight of a 2-hydroxy sulfobetaine of cinnamidoalkyl amine compound having general formula:

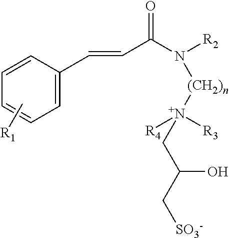

wherein,
R₁ is a substituent, selected from hydrogen, halo, —OH, —NH₂, —NO₂, —OCH₃, —N(CH₃)₂, alkyl groups containing from 1 to 6 carbon atoms, alkoxy groups containing from 1 to 6 carbon atoms, alkylamino or N,N-dialkylamino groups containing from 1 to 6 carbon atoms;
R₂ is selected from hydrogen, alkyl group containing from 1 to 12 carbon atoms;
R₃ and R₄ are independently selected from benzyl, alkyl group containing from 1 to 12 carbon atoms,
n is an integer from 1 to 6,
with the proviso that the UVB sunscreen agents exclude Ethylhexyl Methoxycinnamate and Phenylbenzimidazole Sulfonic Acid.

2-hydroxy sulfobetaine of cinnamidoalkyl amine compound of the present invention is a substantive, water-soluble, UV-B absorbing sunscreen active having general formula:

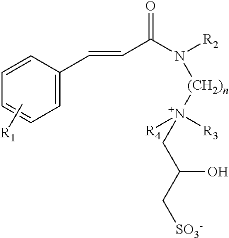

This compound is disclosed in U.S. Pat. No. 6,531,628 assigned to Galaxy Surfactants Ltd.

The preferred 2-hydroxy sulfobetaine of cinnamidoalkyl amine compound is 3-(N-p-Methoxycinnamidopropyl, N,N-Dimethyl Ammonium)-2-Hydroxypropane-1-Sulphonate having the formula:

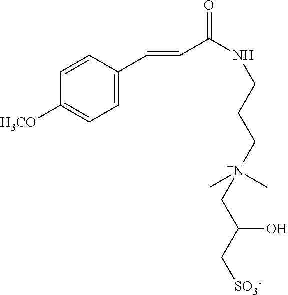

and is commercially available under the name Galaxy SunBeat from the applicant.

Sunscreen agents, active in UV-A and/or UV-B regions, used for the present invention can be water-soluble, fat-soluble or insoluble in commonly used cosmetic solvents. UV-filter substance is preferably selected from conventional UVA and UVB and/or broad spectrum UV-filter substances known to be added into topical compositions such as cosmetic or dermatological sun care products. Such UV-filter substances comprise all groups which absorb light predominantly in the range of wavelengths 400 nm to 320 nm (UVA) and 320 nm to 280 nm (UVB) and which are or can be used as cosmetically acceptable UV-filter substances. Such UV-filter substances are e.g. listed in the PCPC Cosmetic Ingredient Dictionary & Handbook, 13th Edition (2010) or “The Encyclopedia of Ultraviolet Filters” (ISBN: 978-1-932633-25-2) by Nadim A. Shaath.

The UVA-absorbing sunscreen agents are chosen in particular from Dibenzoylmethane compounds.

UVA-absorbing Dibenzoylmethane compound used in the present invention include, but are not limited to, those selected from the group consisting of 2-Methyldibenzoylmethane, 4-Methyldibenzoylmethane, 4-Isopropyldibenzoylmethane, 4-tert-Butyldibenzoylmethane, 2,4-Dimethyldibenzoylmethane, 2,5-Dimethyldibenzoylmethane, 4,4′-Diisopropylbenzoylmethane, 4-tert-Butyl-4′-Methoxydibenzoylmethane, 2-Methyl-5-Isopropyl-4′-Methoxydibenzoylmethane, 2-Methyl-5-tert-Butyl-4′-Methoxydibenzoylmethane, 2,4-Dimethyl-4′-Methoxydibenzoylmethane, 2,6-Dimethyl-4′tert-Butyl-4′Methoxydibenzoylmethane, and mixtures thereof. Other Dibenzoylmethane compounds described as UV-A filters are disclosed in U.S. Pat. Nos. 4,489,057, 4,387,089 and 4,562,067. Preferred UVA-absorbing Dibenzoylmethane compounds include those selected from the group consisting of 4-tert-Butyl-4′-Methoxydibenzoylmethane, 4-Isopropyldibenzoylmethane, and mixtures thereof. A more preferred UVA-absorbing Dibenzoylmethane compound is 4-tert-Butyl-4′-Methoxydibenzoylmethane.

The sunscreen active, 4-tert-Butyl-4′-Methoxydibenzoylmethane, which is also known as Butyl Methoxydibenzoylmethane or Avobenzone having the formula

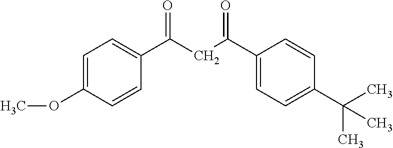 [SMILES: COc1ccc(C(=O)CC(=O)c2ccc(C(C)(C)C)cc2)cc1]

is commercially available under the names GalSORB Avobenzone from Galaxy Surfactants Ltd. (India).

The UVB-sunscreen agents are chosen from Ethylhexyl Salicylate, Homosalate, Isoamyl p-Methoxy cinnamate, Octocrylene, 4-Methylbenzylidene Camphor, 3-Benzylidene camphor, Benzylidene Camphor Sulfonic Acid, Camphor Benzalkonium Methosulfate, Polyacrylamidomethyl Benzylidene Camphor, Ethylhexyl Triazone, Diethylhexyl Butamido Triazone and Polysilicon-15.

The preferred p-aminobenzoic acid derivative, is PABA, Ethyl PABA, Ethyl Dihydroxypropyl PABA, Ethylhexyl Dimethyl PABA sold under the name “Escalol 507” by ISP, DimethylPABAminopropyl Laurdimonium Tosylate sold by the name “Escalol HP-610” by ISP, Glyceryl PABA, PEG-25 PABA sold under the name “Uvinul P25” by BASF,

The preferred Salicylic derivative is Homosalate sold under the name “Eusolex HMS” by Rona/EM Industries, Ethylhexyl Salicylate sold under the name “Neo Heliopan TS” by Haarmann&Reimer, Dipropyleneglycol Salicylate sold under the name “Dipsal” by Scher, TEA Salicylate sold under the name “Neo Heliopan TS” by Haarmann&Reimer, and Isopropylbenzyl Salicylate

The preferred Cinnamic derivative is Ethylhexyl Methoxycinnamate and Methoxycinnamidopropyl Laurdimonium Tosylate sold in particular under the trademarks “GalSORB OMC(HP)” and “Galaxy TosyQuat” respectively by Galaxy Surfactants Ltd., Isopropyl Methoxycinnamate, Isoamyl p-Methoxycinnamate sold under the trademark “Neo Heliopan E 1000” by Haarmann&Reimer, Cinoxate, DEA Methoxycinnamate, Diisopropyl Methylcinnamate, Glyceryl Ethylhexanoate Dimethoxycinnamate,
The preferred β,β.-Diphenylacrylate derivative is Octocrylene sold in particular under the trademark “GalSORB Octocrylene” by Galaxy Surfactants Ltd., Etocrylene sold in particular under the trademark “Uvinul N35” by BASF,

The preferred Benzophenone derivative is Benzophenone-1 sold under the trademark “Uvinul 400” by BASF, Benzophenone-2 sold under the trademark “Uvinul D50” by BASF, Benzophenone-3 or Oxybenzone sold under the trademark “GalSORB Benzophenone-3” by Galaxy Surfactants Ltd., Benzophenone-4 sold under the trademark “GalSORB Benzophenone-4” by Galaxy Surfactants Ltd., Benzophenone-5, Benzophenone-6 sold under the trademark “Helisorb 11” by Norquay, Benzophenone-8 sold under the trademark “Spectra-Sorb UV-24” by American Cyanamid, Benzophenone-9 sold under the trademark “Uvinul DS-49” by BASF, Benzophenone-12,

The preferred Benzylidenecamphor derivative is 4-Methylbenzylidene Camphor manufactured under the name “Neo Heliopan MBC” by Haarmann&Reimer, 3-Benzylidene camphor manufactured under the name “Mexoryl SD” by Chimex, Benzylidene Camphor Sulfonic Acid manufactured under the name “Mexoryl SL” by Chimex, Camphor Benzalkonium Methosulfate manufactured under the name “Mexoryl SO” by Chimex, Terephthalyidene Dicamphor Sulfonic Acid manufactured under the name “Mexoryl SX” by Chimex, Polyacrylamidomethyl Benzylidene Camphor manufactured under the name “Mexoryl SW” by Chimex,

The preferred Triazine derivative is bis-Ethylhexyloxyphenol Methoxyphenyl Triazine, sold under the trademark “Tinosorb S” by Ciba Specialty Chemicals, Ethylhexyl Triazone sold in particular under the trademark “Uvinul T150” by BASF, Diethylhexyl Butamido Triazone sold under the trademark “Uvasorb HEB” by Sigma 3V, 2,4,6-tris(Diisobutyl 4′-Aminobenzalmalonate)-s-Triazine,

The preferred Phenylbenzotriazole derivative is Drometrizoletrisiloxane sold under the name “Mexoryl XL” by Chimex, Methylene bis-Benzotriazolyl Tetramethylbutylphenol sold in the solid form under the trademark “Mixxim BB/100” by Fairmount Chemical or in the micronized form in aqueous dispersion under the trademark “Tinosorb M” by Ciba Specialty Chemicals,

The preferred Anthranilic derivative is Menthyl Anthranilate, sold under the trademark “Neo Heliopan MA” by Haarmann &Reimer,

The preferred Imidazole Derivative is Phenylbenzimidazole Sulfonic Acid, sold under the name “Neo Heliopan Hydro” by Haarmann&Reimer, Disodium Phenyl Dibenzimidazole

Tetrasulfonate, sold under the name “Neo Heliopan AP” by Haarmann&Reimer, Ethylhexyl Dimethoxybenzylidene Dioxoimidazoline Propionate,

The preferred benzalmalonate derivative is Polysilicon-15 (Dimethicodiethylbenzalmalonate) sold under the name “Parsol SLX” by DSM,

The preferred Benzoic acid Derivative is Diethylaminohydroxybenzoylhexylbenzoate, sold under the name “Uvinul A-plus” by CIBA,

The preferred 4,4-diarylbutadiene derivative is 1,1-Dicarboxy (2,2′-Dimethylpropyl)-4,4-Diphenylbutadiene and their mixtures.

Inorganic UV-filter substances encompass pigments or alternatively nanopigments (mean size of the primary particles: generally between 5 nm and 200 nm, preferably between 10 nm and 140 nm) formed from coated or uncoated metal oxides, such as, for example, nanopigments formed from Titanium Oxide (amorphous or crystallized in the rutile and/or anatase form), Iron Oxide, Zinc Oxide, Zirconium Oxide or Cerium Oxide, which are all UV photoprotective agents well known per se. Conventional coating agents are, for example alumina, aluminum stearate, aluminium hydroxoide, silica, dimethicone, simethicone, silanes and stearic acid. Examples for Titanium Dioxide used as UV-filter are “Eusolex T2000”, sold by Merck, “SUNSIL Tin50”, sold by Impag, “Tioveil 50 FIN”, sold by Croda and “Parsol TX” sold by DSM, Examples for Zinc Oxide used as UV filter are “SUNZNO” sold by Impag, “ZinClear IM”, sold by Dow Chemicals and “Solaveil CZ-100”, sold by Croda.

The inorganic UV screening agents which are more particularly preferred are chosen from Titanium Dioxide and Zinc Oxide.

The total amount of 2-hydroxy sulfobetaine of cinnamidoalkyl amine compound, in particular of Galaxy SunBeat, in the topical compositions according to the invention should be at least 0.1 wt % of the total weight of the composition. The preferable range is from about 0.1 to about 20 wt %, in particular in the range of about 2 to 15 wt %, most particular in the range of about 4 to 10 wt %, with respect to the total weight of the topical composition.

The total amount of sunscreen agents in the topical compositions according to the invention is preferably selected in the range of about 0.1 to 25 wt %, in particular in the range of about 2 to 20 wt %, with respect to the total weight of the topical composition.

The compositions of the present invention comprise a carrier, or vehicle, suitable for application to human skin. Such carriers are well-known to one of ordinary skill in the art, and can include one or more compatible liquid or solid filler diluents or vehicles which are suitable for application to human skin. Carrier and its components are suitable for use in contact with human skin without undue toxicity, incompatibility, instability, allergic response, and the like within the scope of sound medical or formulator's judgment. The carrier may comprise one or more active or inactive materials, including but not limited to optional components described below. The carrier comprises the balance of the composition. The compositions of the present invention preferably comprise from about 74% to about 99.7%, more preferably from about 79% to about 99%, carrier by weight of the composition.

The carrier can be formulated in a number of ways, including but not limited to emulsions (in emulsion technology, a composition comprising a “dispersed phase” and a “continuous phase;” the dispersed phase existing as small particles or droplets that are suspended in and surrounded by a continuous phase).

The compositions of the present invention can be formulated into a wide variety of product types, including creams, lotions, milks, mousses, gels, oils, tonics, sticks, and sprays. The compositions of the present invention may contain a variety of other ingredients such as are conventionally used in a given product type provided that they do not unacceptably alter the benefits of the invention. Examples of these ingredient classes include: abrasives, absorbents, aesthetic components such as fragrances, pigments, colorings/colorants, essential oils, skin sensates, astringents, etc., anti-acne agents, anti-aging agents, anti-caking agents, antifoaming agents, additional antimicrobial agents, antioxidants, binders, biological additives, buffering agents, bulking agents, chelating agents, chemical additives, colorants, cosmetic astringents, cosmetic biocides, denaturants, drug astringents, external analgesics, film formers or materials, humectants, opacifying agents, pH adjusters, propellants, reducing agents, sequestrants, skin bleaching agents, skin-conditioning agents, skin soothing and/or healing agents, skin treating agents including agents for preventing, retarding, arresting, and/or reversing skin wrinkles (e.g., alpha-hydroxy acids such as lactic acid and glycolic acid and beta-hydroxy acids such as salicylic acid), thickeners, and vitamins and derivatives thereof (e.g. tocopherol, tocopherol acetate, beta carotene, retinoic acid, retinol, retinoids, retinyl palmitate, niacin, niacinamide, and the like). Such and other cosmetic ingredients commonly used in the personal care industry, which are suitable for use in the compositions of the present invention are e.g. described in the PCPC Cosmetic Ingredient Dictionary & Handbook, 13th Edition (2010) without being limited thereto.

These additional cosmetic or personal care or dermatological ingredients, adjuvants and additives and/or their amounts can, based on the desired product, easily be chosen by a skilled person in this field, of course, by taking care that such ingredients, adjuvants and additives and/or their amounts does not detrimentally affect the advantageous properties intrinsically attached to the inventive concept of the present invention.

The sunscreen composition of the present invention provides strong protection against both UV-B and UV-A rays. SPF (Sun-burn Protection Factor) is an in-vivo method used to measure the protection against sunburn. The higher the SPF, the better is the protection from UV-B radiation. With respect to UV-A protection, the most common test methods used are the in-vivo PPD test for US sunscreens, the in-vitro critical wavelength method (CW), which is the basis for the labeling of the UVA-protection level in the EU

The 2-hydroxy sulfobetaine of cinnamidoalkyl amine compound is added into cosmetic products without a neutralization step. The detailed description on this neutralization and other formulation ease is mentioned in the article titled “water-soluble solution” published by the applicant in SPC Asia Magazine, November 2003, pages 24-26.

Intensity of protection from UV radiation also depends upon the amount of sunscreen filters used in the formulation. SPF of the formulation can be enhanced if the amount of sunscreen filter(s) in the formulation is increased. The incorporation of 2-hydroxy sulfobetaine of cinnamidoalkyl amine compound at a high level of upto 20% by weight helps to formulate high SPF sunscreen products than using Phenylbenzimidazole Sulfonic Acid

Thus, it is possible through present invention to formulate cosmetic products with a synergistic high SPF due to the presence of oil-soluble and water-soluble sunscreens even in the pH-range of 4 to 6, which is the natural pH-range of human skin, meaning a big advantage to the prior state-of-the-art.

In order to further illustrate the present invention and the advantages thereof, the following specific examples are given, it being understood that same are intended only as illustrative and in nowise limitative, as many variations thereof are possible without departing from the spirit and scope of the invention. All the experimental analyses performed herein are conducted in-vitro unless specified else.

### EXAMPLES

The following sunscreen/cosmetic/dermatological products are representative of the present invention.

### Example 1 to 4

### Sunscreen Cream

Sunscreen creams using Galaxy SunBeat were prepared and the efficiency and effectiveness of Galaxy SunBeat was compared with Ethylhexyl Methoxycinnamate and Phenylbenzimidazole Sulfonic Acid. Example 1 was formulated using Octocrylene and Avobenzone, Example 2 was formulated using Galaxy SunBeat alongwith Octocrylene and Avobenzone, Example 3 using Phenylbenzimidazole Sulfonic Acid alongwith Octocrylene and Avobenzone & Example 4 was formulated using GalSORB OMC (Ethylhexyl Methoxycinnamate) along with Octocrylene and Avobenzone.

| | % wt. | | | |
|---|---|---|---|---|
| Ingredients | Ex. 1 | Ex. 2 | Ex. 3 | Ex. 4 |
| Paraffin Oil | 4.0 | 4.0 | 4.0 | 4.0 |
| Stearic acid | 2.0 | 2.0 | 2.0 | 2.0 |
| Glyceryl Mono stearate | 5.0 | 5.0 | 5.0 | 5.0 |
| Cetostearyl alcohol | 3.5 | 3.5 | 3.5 | 3.5 |
| Isopropyl myristate | 5.0 | 5.0 | 5.0 | 5.0 |
| Glycerin | 2.0 | 2.0 | 2.0 | 2.0 |
| Galaxy-MW259 | 0.5 | 0.5 | 0.5 | 0.5 |
| (Lauryl alcohol 9 EO) | | | | |
| Galaxy PEG-7GC | 2.0 | 2.0 | 2.0 | 2.0 |
| (PEG-7 glyceryl cocoate) | | | | |
| Disodium EDTA | 0.1 | 0.1 | 0.1 | 0.1 |
| Galguard NK1 (Blend of Parabens | 0.6 | 0.6 | 0.6 | 0.6 |
| & Phenoxyethanol) | | | | |
| GalSORB Octocrylene | 5.0 | 5.0 | 5.0 | 5.0 |
| GalSORB Avobenzone | 3.0 | 3.0 | 3.0 | 3.0 |
| Galaxy SunBeat | — | 4.0 | — | — |
| PBSA | — | — | 4.0 | — |
| (Phenylbenzimidazole Sulfonic Acid) | | | | |
| GalSORB OMC | — | — | — | 4.0 |
| (Ethylhexyl Methoxycinnamate) | | | | |
| Distilled water | 67.3 | 58.2 | 63.3 | 63.3 |
| Total | 100 | 100 | 100 | 100 |

The resulting sunscreen creams were tested for UVA PF, Critical wavelength (CW), on Optometrics SPF meter (Model 290 S equipped with Xenon lamp).

Procedure:

A Transpore tape (Ready to use VITRO substrate provided by Optometrics, USA), IN VITRO skin substrate was used for testing the sunscreen formulations. To prepare slides for testing, sunscreen cream was drawn in a syringe and applied on Transpore tape (stretched on sample holder) in the form of very small dots. The amount of test sample utilized was sufficient to dose the area at 2 mg/cm² (or 0.1 gm to the 50 cm² area). The sunscreen test sample was uniformly spread over a 50 cm² area, with a finger cot on. The dots were spread out into as uniform a thickness as possible by spreading alternatively vertically and horizontally. The slide so prepared was allowed to dry for 20 minutes. 5 slides were measured per sample. To measure SPF and other parameters, SPF meter equipped with automatic x-y stage and software calculated all the parameters as an average of 12 scans/runs per slides.

| | UV Filter (% wt.) | | | | | | UVA | |
|---|---|---|---|---|---|---|---|---|
| Ex. | SB | PBSA | OMC | OCN | AVB | SPF | PF | CW |
| 1 | — | — | — | 5.0 | 3.0 | 7.5 | 9.0 | 377.9 |
| 2 | 4.0 | — | — | 5.0 | 3.0 | 18.0 | 14.6 | 377.2 |
| 3 | — | 4.0 | — | 5.0 | 3.0 | 13.1 | 8.4 | 377.6 |
| 4 | — | — | 4.0 | 5.0 | 3.0 | 19.0 | 9.5 | 374.9 |

(Abbreviations: OMC—Ethylhexyl Methoxycinnamate, OCN—Octocrylene, AVB—Avobenzone, PBSA—Phenylbenzimidazole Sulfonic Acid, SB—SunBeat,)

Further formulations with regards to the claimed invention are as follows:

% wt.

| Ingredients | Ex. 5 | Ex. 6 | Ex. 7 | Ex. 8 |
|---|---|---|---|---|
| Paraffin Oil | 4.0 | 4.0 | 4.0 | 4.0 |
| Stearic acid | 2.0 | 2.0 | 2.0 | 2.0 |
| Glyceryl Mono stearate | 5.0 | 5.0 | 5.0 | 5.0 |
| Cetostearyl alcohol | 3.5 | 3.5 | 3.5 | 3.5 |
| Isopropyl myristate | 5.0 | 5.0 | 5.0 | 5.0 |
| Glycerin | 2.0 | 2.0 | 2.0 | 2.0 |
| Galaxy-MW259 | 0.5 | 0.5 | 0.5 | 0.5 |
| (Lauryl alcohol 9 EO) | | | | |
| Galaxy PEG-7GC | 2.0 | 2.0 | 2.0 | 2.0 |
| (PEG-7 glyceryl cocoate) | | | | |
| Disodium EDTA | 0.1 | 0.1 | 0.1 | 0.1 |
| Galaxy Galguard | 0.7 | 0.3 | 0.7 | 0.3 |
| (Blend of Parabens & | | | | |
| Phenoxy ethanol) | | | | |
| Galaxy SunBeat | — | 4.4 | — | 4.4 |
| GalSORB Octocrylene | — | — | 5.0 | 5.0 |
| GalSORB Avobenzone | 3.0 | 3.0 | 3.0 | 3.0 |
| GalSORB OMC | 5.0 | — | 5.0 | — |
| (Ethylhexyl Methoxy | | | | |
| Cinnamate) | | | | |
| Distilled water | 67.2 | 68.2 | 62.2 | 63.2 |
| Total | 100 | 100 | 100 | 100 |

Above formulations were analyzed for SPF, UV-A PF, and C.W. and the results are tabulated as given below:

| | | | | | Total UV | | | |
|---|---|---|---|---|---|---|---|---|
| | UV Filter (% wt.) | | | | Filters | | UVA | |
| Ex. | OMC | OCN | AVB | SB | (% wt.) | SPF | PF | C.W. |
| 5 | 5.0 | — | 3.0 | — | 8.0 | 15.0 | 10.0 | 378 |
| 6 | — | — | 3.0 | 4.4 | 7.4 | 15.0 | 15.0 | 378 |
| 7 | 5.0 | 5.0 | 3.0 | — | 13.0 | 17.4 | 10.0 | 378 |
| 8 | — | 5.0 | 3.0 | 4.4 | 12.4 | 22.0 | 17.0 | 380 |

(Abbreviations: OMC—Octyl methoxycinnamate, OCN—Octocrylene, AVB—Avobenzone, SB—SunBeat, C.W.—Critical Wavelength)

% wt.

| Ingredients | Ex. 9 | Ex. 10 |
|---|---|---|
| Paraffin Oil | 4.0 | 4.0 |
| Stearic acid | 2.0 | 2.0 |
| Glyceryl Mono stearate | 5.0 | 5.0 |
| Cetostearyl alcohol | 3.5 | 3.5 |
| Isopropyl myristate | 5.0 | 5.0 |
| Glycerin | 2.0 | 2.0 |
| Galaxy-MW259 | 0.5 | 0.5 |
| (Lauryl alcohol 9 EO) | | |
| Galaxy PEG-7GC | 2.0 | 2.0 |
| (PEG-7 glyceryl cocoate) | | |
| Disodium EDTA | 0.1 | 0.1 |
| Galaxy Galguard | 0.3 | 0.3 |
| (Blend of Parabens & | | |
| Phenoxy ethanol) | | |
| Galaxy SunBeat | — | 2.6 |
| GalSORB Octocrylene | 4.0 | 4.0 |
| GalSORB Avobenzone | 2.4 | 2.4 |
| PBSA | 2.6 | — |
| (Phenylbenzimidazole | | |
| Sulfonic Acid) | | |
| Benzophenone-3 | 3.2 | 3.2 |
| Distilled water | q.s to 100 | q.s. to 100 |
| Total | 100 | 100 |

Above formulations were analyzed for SPF, UV-A PF, and C.W. and the results are tabulated as given below:

| | | | | | | Total | | | |
|---|---|---|---|---|---|---|---|---|---|
| | | | | | | UV | | | |
| | UV Filter (% wt.) | | | | | Filters | | UVA | |
| Ex. | PBSA | OCN | AVB | B-3 | SB | (% wt.) | SPF | PF | C.W. |
| 9 | 2.6 | 4.0 | 2.4 | 3.2 | — | 12.2 | 15.0 | 12.0 | 377 |
| 10 | — | 4.0 | 2.4 | 3.2 | 2.6 | 12.2 | 25.0 | 21.0 | 377 |

From the above tabulated results it can be inferred that composition containing AVB and OCN gives better UV-A protection in combination with SunBeat than in combination with PBSA and/or OMC

Furthermore, the formulas I to 4 above where tested for photostability by exposing the formulas to sunlight for three hours (10 am to 1 pm, 2 Jun. 2011, Navi Mumbai, INDIA). The remaining content of AVB was measured using UV Spectrophotometer and compared to unexposed control.

| | UV Filter (% wt.) | | | | | % AVB retained after 3 hours |
|---|---|---|---|---|---|---|
| Ex. | SB | PBSA | OMC | OCN | AVB | exposure to Sunlight |
| 1 | — | — | — | 5.0 | 3.0 | 90 |
| 2 | 4.0 | — | — | 5.0 | 3.0 | 92 |
| 3 | — | 4.0 | — | 5.0 | 3.0 | 78 |
| 4 | — | — | 4.0 | 5.0 | 3.0 | 72 |

The results are showing, that photostability of sunscreen containing AVB/OCN is better in combination with SunBeat compared to combination with OMC or PBSA.

Thus, overall results show that SunBeat provides higher UVA protection and enhanced photostability along with good SPF compared to PBSA and OMC.

Following are the few other specific examples illustrating the present invention.

### Example 11

### Sun Lotions o/w

| | 11 |
|---|---|
| Potassium Cetyl Phosphate | 2.0% |
| Cetyl Alkohol | 0.5% |
| Dicaprylyl Carbonate | 5.0% |
| Ethylhexyl Palmitate | 2.0% |
| Caprylic/Capric Triglyceride | 2.0% |
| 3-(N-p-methoxy cinnamidopropyl. N.N- | 3.0% |
| dimethyl ammonium)-2-hydroxy propane-1- | |
| sulphonate (SunBeat) | |
| Butyl Methoxydibenzoylmethane | 1.0% |
| Bis-Ethylhexyloxyphenol Methoxyphenyl | 3.0% |
| Triazine | |
| Drometrizole Trisiloxane | 0.5% |
| Octocrylene | 4.0% |
| Diethylhexyl Butamido Triazone | 4.0% |
| Polysilicon-15 | 0.5% |
| Zinc Oxide | 2.0% |
| Glycerin | 5.0% |
| BHT | 0.1% |
| VP/Hexadecene Copolymer | 1.0% |
| Acrylates/C10-30 Alkyl Acrylate | 0.5% |
| Crosspolymer | |
| Trisodium EDTA | 0.1% |
| Methylpropanediol | 0.2% |
| Phenoxyethanol | 0.5% |
| Methylparaben | 0.3% |
| Sodium Hydroxide, Colourants | q.s. |
| Parfum | q.s. |
| Aqua | ad 100 |

### Example 12

### Sun Lotions o/w

| | 12 |
|---|---|
| Glyceryl Sterarate Citrate | 2.0% |
| Stearyl Alcohol | 0.5% |
| Butylene Glycol Dicaprylate/Dicaprate | 5.0% |
| Dicaprylyl Ether | 2.0% |
| Paraffinum Liquidum | 2.0% |
| 3-(N-p-methoxy cinnamidopropyl. N,N- | 4.0% |
| dimethyl ammonium)-2-hydroxy | |
| propane-1-sulphonate (SunBeat) | |
| Bis-Ethylhexyloxyphenol | 4.0% |
| Methoxyphenyl Triazine | |
| Methylene Bis-Benztriazolyl | 0.5% |
| Tetramethylbutylphenol | |
| Disodium Phenyl Dibenzimidazole | 0.5% |
| Tetrasulfonate | |
| Terephthalidene Dicamphor Sulfonic | 0.5% |
| Acid | |
| Octocrylene | 4.0% |
| Diethylhexyl Butamido Triazone | 4.0% |
| Polysilicon-15 | 0.5% |
| Aluminium Starch | 1.0% |
| Glycerin | 5.0% |
| BHT | 0.1% |
| VP-Eicosene copolymer | 0.8% |
| Acrylates/C10-30 Alkyl Acrylate | 0.1% |
| Crosspolymer | |
| Trisodium EDTA | 0.1% |
| Methylpropanediol | 0.2% |
| Phenoxyethanol | 0.5% |
| Ethylylparaben | 0.3% |
| Sodium Hydroxide. Colourants | q.s. |
| Parfum | q.s. |
| Aqua | ad 100 |

### Example 13

### Sun Lotions o/w

| | 13 |
|---|---|
| Potassium Cetyl Phosphate | 2.0% |
| PEG-100 Stearate | 1.0% |
| Cetearyl Alcohol | 0.5% |
| Cyclopentasiloxane | 1.5% |
| Isohexadecane | 2.0% |
| Paraffinum Liquidum | 2.0% |
| 3-(N-p-methoxy cinnamidopropyl. | 2.0% |
| N,N-dimethyl ammonium)-2-hydroxy | |
| propane-1-sulphonate (SunBeat) | |
| Butyl Methoxydibenzoylmethane | 3.0% |
| Benzophenone-4 | 0.5% |
| Terephthalidene Dicamphor Sulfonic | 0.5% |
| Acid | |
| Octocrylene | 4.0% |
| Butylene Glycol | 2.0% |
| VP-Eicosene copolymer | 0.8% |
| Acrylamid/Sodium | 0.1% |
| Acryloyldimethyltaurate Copolymer | |
| Styrene/Acrylates Copolymer | — |
| Phenoxyethanol | 0.5% |
| Ethylylparaben | 0.3% |
| Sodium Hydroxide, Colourants | q.s. |
| Parfum | q.s. |
| Aqua | ad 100 |

### Example 14

### Sun Creams o/w

| | 14 |
|---|---|
| Cetyl Phosphate | 2.5% |
| Cetyl Alkohol | 0.8% |
| C12-15 Alkyl Benzoate | 6.0% |
| Octyldodecanol | 2.0% |
| Paraffinum Liquidum | 2.0% |
| Beeswax | 1.0% |
| 3-(N-p-methoxy cinnamidopropyl. N,N- | 2.0% |
| dimethyl ammonium)-2-hydroxy | |
| propane-1-sulphonate (SunBeat) | |
| Butyl Methoxydibenzoylmethane | 1.0% |
| Bis-Ethylhexyloxyphenol | 3.0% |
| Methoxyphenyl Triazine | |
| Octocrylene | 4.0% |
| Diethylhexyl Butamido Triazone | 4.0% |
| Titanium Dioxide | 2.0% |
| Glycerin | 5.0% |
| VP/Hexadecene Copolymer | 1.0% |
| Acrylates/C10-30 Alkyl Acrylate | 1.0% |
| Crosspolymer | |
| Carbomer | 0.3% |
| Phenoxyethanol | 0.5% |
| Methylparaben | 0.3% |
| Sodium Hydroxide, Colourants | q.s. |
| Parfum | q.s. |
| Aqua | ad 100 |

### Example 15

### Sun Lotions w/o

| | 15 |
|---|---|
| Polyglyceryl-2 Sesquiisostearate | 2.0% |
| Dicaprylyl Carbonate | 3.0% |
| Paraffinum Liquidum | 5.0% |
| Dimethicone | 2.0% |
| Cera Microcristallina | 1.0% |
| 3-(N-p-methoxy cinnamidopropyl. N,N- | 3.0% |
| dimethyl ammonium)-2-hydroxy | |
| propane-1-sulphonate (SunBeat) | |
| Butyl Methoxydibenzoylmethane | 4.0% |
| Bis-Ethylhexyloxyphenol | 2.0% |
| Methoxyphenyl Triazine | |
| Terephthalidene Dicamphor Sulfonic | 0.5% |
| Acid | |
| Octocrylene | 2.0% |
| Diethylhexyl Butamido Triazone | 4.0% |
| Polysilicon-15 | 0.5% |
| Titanium Dioxide | 3.0% |
| Butylene Glycol | 5.0% |
| Tocopheryl Acetate | 0.5% |
| Hexandiol | 0.2% |
| Phenoxyethanol | 1.0% |
| Propylparaben | 0.4% |
| Sodium Hydroxide, Colourants | q.s. |
| Parfum | q.s. |
| Aqua | ad 100 |

### Example 16-17

### Sun Sprays

| | 16 | 17 |
|---|---|---|
| C12-15 Alkyl Benzoate | 1.0% | 3.0% |
| Cyclomethicone | 4.0% | 2.0% |
| 3-(N-p-methoxy cinnamidopropyl. N,N- | 3.0% | 2.0% |
| dimethyl ammonium)-2-hydroxy propane- | | |
| 1-sulphonate (SunBeat) | | |
| Butyl Methoxydibenzoylmethane | 3.0% | — |
| Bis-Ethylhexyloxyphenol Methoxyphenyl | — | 3.0% |
| Triazine | | |
| Diethylaminohydroxybenzoylhexylbenzoate | — | 2.0% |
| Benzophenone-3 | 1.0% | — |
| Homosalate | 4.0% | — |
| Ethylhexyl Salicylate | 2.0% | — |
| Octocrylene | 3.0% | 5.0% |
| Ethylhexyl Triazone | — | 2.5% |
| Acrylates/C10-30 Alkyl Acrylate | 0.8% | — |
| Crosspolymer | | |
| Acrylates/Octylacrylamide Copolymer | — | 0.7% |
| Sodium Hydroxide, Colourants | q.s. | q.s. |
| Parfum | q.s. | q.s. |
| Alcohol | ad | ad |
| | 100 | 100 |

### Example 18-19

### Sun Sprays

| | 18 | 19 |
|---|---|---|
| Acrylates/C10-30 Alkyl Acrylate | — | 0.5% |
| Crosspolymer | | |
| Acrylates Copolymer | 0.5% | — |
| Dilinoleate Copolymer | 0.5% | 0.5% |
| C12-15 Alkyl Benzoate | 2.0% | 3.0% |
| C18-36 Triglycerides | 1.5% | — |
| Cyclomethicone | 1.5% | 2.0% |
| 3-(N-p-methoxy cinnamidopropyl. N,N- | 2.0% | 3.0% |
| dimethyl ammonium)-2-hydroxy propane- | | |
| 1-sulphonate (SunBeat) | | |
| Butyl Methoxydibenzoylmethane | 4.0% | — |
| Bis-Ethylhexyloxyphenol Methoxyphenyl | — | 2.0% |
| Triazine | | |
| Diethylaminohydroxybenzoylhexylbenzoate | — | 1.0% |
| Terephthalidene Dicamphor Sulfonic Acid | — | 0.5% |
| Drometrizole Trisiloxane | — | 0.5% |
| Benzophenone-3 | 1.0% | — |
| Homosalate | 4.0% | — |
| Ethylhexyl Salicylate | 2.0% | — |
| Octocrylene | 3.0% | 5.0% |
| Ethylhexyl Triazone | — | 2.5% |
| Titanium Dioxide | — | 0.5% |
| Zinc Oxide | 2.0% | — |
| Glycerin | 5.0% | — |
| Alcohol | — | 3.0% |
| VP/Hexadecene Copolymer | 0.5% | 0.5% |
| Methylpropanediol | — | — |
| Ethylhexylglycerin | — | 0.5% |
| Phenoxyethanol | 1.0% | 0.8% |
| Methylparaben | 0.5% | 0.4% |
| Methylisothiazolinone | 0.3% | — |
| DMDM Hydantoin | — | — |
| Sodium Hydroxide, Colourants | q.s. | q.s. |
| Parfum | q.s. | q.s. |
| Aqua | ad | ad |
| | 100 | 100 |

### Example 20 to 21

### Face Creams

| | 20 | 21 |
|---|---|---|
| PEG-100 Stearate | 2.0% | 2.5% |
| Glyceryl Sterarate | 1.0% | 0.5% |
| Cetyl Alkohol | 0.5% | — |
| Stearyl Alcohol | — | 0.5% |
| Myristyl Myristate | 5.0% | 3.0% |
| C12-15 Alkyl Benzoate | — | 2.0% |
| Paraffinum Liquidum | 2.0% | — |
| Caprylic/Capric Triglyceride | — | 3.0% |
| Cyclopentasiloxane | 2.0% | 2.0% |
| Isohexadecane | 1.0% | 1.0% |
| Carnauba Wax | 0.5% | — |
| Mica | 1.0% | — |
| Nylon-12 | — | 1.0% |
| 3-(N-p-methoxy cinnamidopropyl. | 1.0% | 1.0% |
| N,N-dimethyl ammonium)-2- | | |
| hydroxy propane-1-sulphonate | | |
| (SunBeat) | | |
| Butyl Methoxydibenzoylmethane | 1.0% | 1.0% |
| Benzophenone-3 | 0.3% | — |
| Drometrizole Trisiloxane | — | 0.5% |
| Homosalate | — | — |
| Ethylhexyl Salicylate | — | 1.0% |
| Octocrylene | 2.0% | — |
| Diethylhexyl Butamido Triazone | 2.0% | 2.0% |
| Phenylbenzimidazole Sulfonic Acid | — | — |
| Titanium Dioxide | — | 1.5% |
| Glycerin | 5.0% | — |
| Acrylamide/Sodium | 0.5% | 0.3% |
| Acryloyldimethyltaurate Copolymer | | |
| Acrylates Copolymer | — | 0.2% |
| Alcohol | 2.0% | 1.0% |
| Hexandiol | — | 0.1% |
| Phenoxyethanol | 0.5% | 0.5% |
| Methylparaben | 0.3% | — |
| DMDM Hydantoin | — | 0.1% |
| Sodium Hydroxide, Colourants | q.s. | q.s. |
| Parfum | q.s. | q.s. |
| Aqua | ad 100 | ad 100 |

### Example 22 to 23

### Face Creams

| | 22 | 23 |
|---|---|---|
| Sucrose Polystearate | 2.0% | — |
| Polyglyceryl-3 Methylglucose | — | 2.5% |
| Distearate | | |
| Myristyl Myristate | 5.0% | 3.0% |
| C12-15 Alkyl Benzoate | — | 2.0% |
| Paraffinum Liquidum | 2.0% | — |
| Caprylic/Capric Triglyceride | — | 3.0% |
| Dimethicone | 2.0% | 2.0% |
| Isohexadecane | 1.0% | 1.0% |
| Beeswax | 0.5% | — |
| Mica | 1.0% | — |
| Tapioca Starch | — | 1.0% |
| 3-(N-p-methoxy cinnamidopropyl. | 1.0% | 1.0% |
| N,N-dimethyl ammonium)-2- | | |
| hydroxy propane-1-sulphonate | | |
| (SunBeat) | | |
| Butyl Methoxydibenzoylmethane | 1.0% | 1.0% |
| Ethylhexyl Salicylate | — | 1.0% |
| Octocrylene | 2.0% | — |
| Diethylhexyl Butamido Triazone | 2.0% | 2.0% |
| Titanium Dioxide | — | 1.5% |
| Glycerin | 5.0% | — |
| Ammonium | — | 0.3% |
| Acryloyldimethyltaurate/VP | | |
| Copolymer | | |
| Acrylates/C10-30 Alkyl Acrylate | 0.2% | — |
| Crosspolymer | | |
| Carbomer | 0.1% | 0.2% |
| Alcohol | 2.0% | — |
| Ethylhexylglycerin | — | 0.1% |
| Phenoxyethanol | 0.5% | 0.5% |
| Methylparaben | 0.3% | — |
| DMDM Hydantoin | — | 0.1% |
| Sodium Hydroxide, Colourants | q.s. | q.s. |
| Parfum | q.s. | q.s. |
| Aqua | ad 100 | ad 100 |

This invention may be embodied in other forms or carried out in other ways without departing from the spirit or essential characteristics thereof. The embodiments given hereinbefore are therefore to be considered as in all respects illustrative and not restrictive, the scope of the invention being indicated by the appended claims, and all changes which come within the meaning and range of equivalency are intended to be embraced therein.

1. A photostable cosmetic composition for protection against UVA and UVB radiations of wavelengths between 280 and 400 nm, which comprises, in a cosmetically acceptable vehicle;
(i) at least one UVA-sunscreen agents selected from the group consisting of Butyl Methoxydibenzoylmethane/Avobenzone, Benzophenone-3, Benzophenone-4, Benzophenone-5, Disodium Phenyl Dibenzimidazole Tetrasulfonate, Drometrizoletrisiloxane, Terephthalyidene Dicamphor Sulfonic Acid, bis-Ethylhexyloxyphenol Methoxyphenyl Triazine, Methylene bis Benzotriazolyl Tetramethylbutylphenol, and Diethylaminohydroxybenzoylhexylbenzoate in the range 0.5 to 5%;
(ii) at least one UVB-sunscreen agents selected from the group consisting of Ethylhexyl Salicylate, Homosalate, Isoamyl p-Methoxy cinnamate, Octocrylene, 4-Methylbenzylidene Camphor, 3-Benzylidene camphor, Benzylidene Camphor Sulfonic Acid, Camphor Benzalkonium Methosulfate, Polyacrylamidomethyl Benzylidene Camphor, Ethylhexyl Triazone, Diethylhexyl Butamido Triazone and Polysilicon-15 in the range 0.5 to 10% and
(iii) at least 0.1% by weight of a 2-hydroxy sulfobetaine of cinnamidoalkyl amine compound having general formula: 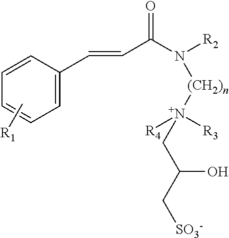
wherein,
R₁ is a substituent, selected from hydrogen, halo, —OH, —NH₂, —NO₂, —OCH₃, —N(CH₃)₂, alkyl groups containing from 1 to 6 carbon atoms, alkoxy groups containing from 1 to 6 carbon atoms, alkylamino or N,N-dialkylamino groups containing from 1 to 6 carbon atoms;
R₂ is selected from hydrogen, alkyl group containing from 1 to 12 carbon atoms;
R₃ and R₄ are independently selected from benzyl, alkyl group containing from 1 to 12 carbon atoms,
n is an integer from 1 to 6.
with the proviso that the UVB sunscreen agents exclude Ethylhexyl Methoxycinnamate and Phenylbenzimidazole Sulfonic Acid.

2. The composition according to claim 1, wherein it comprises from about 0.1 to 20% by weight of a 2-hydroxy sulfobetaine of cinnamidoalkyl amine compound

3. The composition according to claim 1, wherein the 2-hydroxy sulfobetaine of cinnamidoalkyl amine compound is 3-(N-p-Methoxy cinnamidopropyl, N,N-dimethyl ammonium)-2-hydroxy propane-1-sulphonate.

4. The composition according to claim 1 wherein the UVA-sunscreen agent and the 2-hydroxy sulfobetaine of cinnamidoalkyl amine compound is present in the ratio 1:1.5.

5. The composition according to claim 1 wherein the UVB-sunscreen agent and the 2-hydroxy sulfobetaine of cinnamidoalkyl amine compound is present in the ratio 1:1.

6. The composition according to claim 1 containing additionally pigments or nanopigments formed of metal oxides which are coated or non-coated.

7. The composition according to claim 6 where the pigments or nanopigments are selected from the Oxides of Titanium, Zinc, Iron, Zirconium, and Cerium.

8. The composition according to claim 1, wherein the composition is provided in the form of an emulsion, in particular a o/w-emulsion, a lotion, a cream, a spray, a stick, a gel, a mousse, and an oil.
